(19) United States
(12) Patent Application Publication
(10) Pub. No.: US 2025/0290914 A1
(43) Pub. Date: Sep. 18, 2025

(54) DEVICE AND METHOD FOR MEASURING PETROLEUM IN LARGE DIAMETER, ELECTRONIC DEVICE AND MEASURING SYSTEM

(71) Applicants: Chengdu Sea Pioneers Technology Co., LTD., Chengdu (CN); Wuxi Sea Pioneers Technology Co., Ltd., Wuxi (CN)

(72) Inventors: Haibo LIANG, Chengdu, Sichuan Province (CN); Jige CHEN, Chengdu (CN)

(21) Appl. No.: 18/808,113

(22) Filed: Aug. 19, 2024

(30) Foreign Application Priority Data
Mar. 14, 2024 (CN) 2024102898681

(51) Int. Cl. G01N 33/28; G01N 23/12

(52) U.S. Cl. CPC G01N 33/2823; G01N 23/12; G01N 33/2847

(57) ABSTRACT

A method for measuring petroleum in a large diameter, an electronic device and a measuring system are provided. The device for measuring in a large diameter includes: a measurement tube; and a mounting bracket located inside the measurement tube, wherein the mounting bracket is provided with a plurality of photon quantum sources, each photon quantum source is capable of emitting multiple kinds of photon quanta with different energies, and a gap for a to-be-measured petroleum to flow through is reserved between each photon quantum source and an inner wall of the measurement tube; and an outer wall of the measurement tube is correspondingly provided with a photon quantum sensor for each photon quantum source, for detecting multiple kinds of photon quanta emitted by corresponding photon quantum source.

### CROSS-REFERENCE TO RELATED APPLICATION

The present disclosure claims the priority to the Chinese patent application with the filing No. 2024102898681 filed on Mar. 14, 2024 with the China National Intellectual Property Administration and entitled “DEVICE AND METHOD FOR MEASURING PETROLEUM IN LARGE DIAMETER, ELECTRONIC DEVICE AND MEASURING SYSTEM”, the contents of which are incorporated by reference herein in entirety.

### TECHNICAL FIELD

The present disclosure relates to the field of phase fraction measurement of petroleum, and specifically to a device and a method for measuring petroleum in a large diameter, an electronic device and a measuring system.

### BACKGROUND ART

In petroleum domain, accurate measurement of its relevant phase fraction is of great significance. For example, currently, in the petroleum transition, a water cut of the crude oil is measured. The lower the water cut, the better the quality of the crude oil. A minus error of the water cut is likely to differ in the amount of the transitioned petroleum, and further bring a huge economic loss. Therefore, the accuracy of the water cut resulting in important economic losses.

Restricted by penetration capability of an exemption-level photon quantum source (also called as isotope source), currently for scenes of measuring the petroleum in a large diameter, a controlled gamma radioactive source with high energy and activity is generally required for measurement, for example, controlled radioactive source Cs137 with an energy of 660 k eV. During this measurement, the radioactive source is provided on one side of pipeline, a gamma probe (also called a gamma ray sensor) is provided on opposite side of pipeline to detect gamma rays, and phase fraction measurement is performed on the crude oil in the pipeline according to attenuation characteristics of the gamma rays. However, when the controlled radioactive source is used for the phase fraction measurement, the procurement, import and export, transportation, storage, off-site use, installation, wiping and testing and so on of the radioactive source have large management costs and radiation hazard risks. Therefore, how to adopt the exemption-level photon quantum source to carry out high-precision measurement in large-diameter scenes has become an urgent problem to be solved in the field.

### SUMMARY

In order to overcome the shortcomings in the prior art, the present disclosure provides a device and a method for measuring petroleum in a large diameter, an electronic device and a measuring system, specifically including the following.

In the first aspect, the present disclosure provides a device for measuring petroleum in a large diameter. The device for measuring petroleum in a large diameter includes:
a measurement tube; and
a mounting bracket, located inside the measurement tube, wherein the mounting bracket is provided with a plurality of photon quantum sources, and a gap for a to-be-measured crude oil product to flow through is reserved between each of the photon quantum sources and an inner wall of the measurement tube; and
each of the photon quantum sources is capable of emitting multiple kinds of photon quanta, the multiple kinds of photon quanta have different energies, and an outer wall of the measurement tube is provided with photon quantum sensors for the respective photon quantum sources, for detecting the multiple kinds of photon quanta emitted by corresponding photon quantum sources.

In combination with an optional implementation mode of the first aspect, the mounting bracket includes:
a mounting member, wherein each of the photon quantum sources is provided on the mounting member; and
a fixing member, wherein the fixing member is configured for fixedly connecting the mounting member to the inner wall of the measurement tube.

In combination with an optional implementation mode of the first aspect, the mounting member includes at least one mounting rod and an arrangement member;
the arrangement member is arranged coaxially with the measurement tube; and
one end of each mounting rod is connected to the arrangement member, and one end of each mounting rod away from the arrangement member is provided with one of the photon quantum sources.

In combination with an optional implementation mode of the first aspect, the mounting member includes a mounting ring parallel to a cross section of the measurement tube; and
each of the photon quantum sources is provided at a periphery of the mounting ring.

In the second aspect, the present disclosure further provides a method for measuring petroleum in a large diameter, applied to the device for measuring petroleum in a large diameter. The method includes steps of:
obtaining, by the photon quantum sensor, a photon quantum count of each kind of photon quantum;
determining, according to the photon quantum count of each kind of the photon quanta respectively, attenuation intensity of each kind of the photon quantum generated by the to-be-measured petroleum; and
determining, if attenuation intensities of multiple kinds of photon quanta satisfy a preset constraint relationship, phase fraction information about the to-be-measured petroleum according to photon quantum counts of the multiple kinds of photon quanta.

In combination with an optional implementation mode of the second aspect, the step of determining if the attenuation intensities of the multiple kinds of photon quanta satisfy the preset constraint relationship phase fraction information about the to-be-measured petroleum according to photon quantum counts of the multiple kinds of photon quanta includes a step of:
determining, if the attenuation intensities of the multiple kinds of photon quanta shows that the larger the energy of the photon quantum is, the smaller the attenuation is, the phase fraction information about the to-be-measured petroleum according to the photon quantum counts of the multiple kinds of photon quanta.

In combination with an optional implementation mode of the second aspect, the phase fraction information includes an actual water cut of the to-be-measured petroleum, and the step of determining phase fraction information about the to-be-measured petroleum according to photon quantum counts of the multiple kinds of photon quanta includes steps of:
obtaining multiple water cuts of the to-be-measured petroleum according to the photon quantum counts of the multiple kinds of photon quanta; and
determining the actual water cut of the to-be-measured petroleum according to the multiple water cuts.

In combination with an optional implementation mode of the second aspect, the step of obtaining multiple water cuts of the to-be-measured petroleum according to the photon quantum counts of the multiple kinds of photon quanta includes steps of:
determining, according to the photon quantum count of each kind of photon quantum respectively, the water cut corresponding to the photon quantum count of each kind of the photon quantum;
combining the photon quantum counts of the multiple kinds of photon quanta to obtain a plurality of photon quantum combinations; and
determining, according to each of the photon quantum combinations respectively, the water cut corresponding to each of the photon quantum combinations.

In the third aspect, the present disclosure further provides an electronic device, including a processor and a memory, wherein the memory stores a computer program, and the computer program, when executed by the processor, implements the method for measuring petroleum in a large diameter.

In the fourth aspect, the present disclosure further provides a measurement system, including the device for measuring petroleum in a large diameter and the electronic device.

Compared with the prior art, the present disclosure has the following beneficial effects:

The present disclosure provides a device and a method for measuring petroleum in a large diameter, an electronic device and a measuring system. The device for measuring in a large diameter includes: a measurement tube; and a mounting bracket located inside the measurement tube, wherein the mounting bracket is provided with at least one photon quantum source, each photon quantum source is capable of emitting multiple kinds of photon quanta with different energies, and a gap for petroleum to flow through is reserved between each photon quantum source and an inner wall of the measurement tube; and an outer wall of the measurement tube is correspondingly provided with a photon quantum sensor for each photon quantum source, for detecting multiple kinds of photon quanta emitted by corresponding photon quantum source. Since the photon quantum source is fixed inside the measurement tube through the mounting bracket, the photon quanta generated by the photon quantum source can easily penetrate the to-be-measured petroleum and be received by corresponding photon quantum sensor, and thus it is suitable for scenes for measuring petroleum in a large diameter.

### BRIEF DESCRIPTION OF DRAWINGS

In order to more clearly illustrate technical solutions of embodiments of the present disclosure, drawings which need to be used in the embodiments will be briefly introduced below. It should be understood that the drawings merely show some embodiments of the present disclosure, and thus should not be considered as limitation to the scope. Those ordinarily skilled in the art still could obtain other relevant drawings according to these drawings, without using any inventive efforts.

FIG. 1 is a first structural schematic diagram of a device for measuring in a large diameter according to embodiments of the present disclosure;

FIG. 2 shows a longitudinal section of a device for measuring in a large diameter according to embodiments of the present disclosure;

FIG. 3 is a second structural schematic diagram of a device for measuring in a large diameter according to embodiments of the present disclosure;

FIG. 4 is a third structural schematic diagram of a device for measuring in a large diameter according to embodiments of the present disclosure;

FIG. 5 is a schematic flow chart of a method for measuring petroleum in a large diameter according to embodiments of the present disclosure; and

FIG. 6 is a structural schematic diagram of an electronic device provided in embodiments of the present disclosure.

Reference signs: 11—measurement tube; 12—photon quantum sensor; 13—mounting bracket; 14—photon quantum source; 15—electronic device; 131—mounting rod; 132—arrangement member; 133—fixing member; 134—mounting ring; 201—memory; 202—processor; 203—communication unit; 204—system bus.

### DETAILED DESCRIPTION OF EMBODIMENTS

In order to make the objectives, technical solutions and advantages of the embodiments of the present disclosure more clear, the technical solutions in the embodiments of the present disclosure will be described clearly and completely below in conjunction with the drawings of the embodiments of the present disclosure. Apparently, the embodiments described are some but not all embodiments of the present disclosure. Generally, components in the embodiments of the present disclosure described and shown in the drawings herein may be arranged and designed in various different configurations.

Therefore, the following detailed description of the embodiments of the present disclosure provided in the drawings is not intended to limit the claimed scope of the present disclosure, but merely represents selected embodiments of the present disclosure. All of other embodiments obtained by those ordinarily skilled in the art based on the embodiments in the present disclosure without using any inventive efforts shall fall within the scope of protection of the present disclosure.

It should be noted that like reference signs and letters represent like items in the following drawings, and thus, once a certain item is defined in one drawing, it is unnecessary to define and explain the same in subsequent drawings.

In the description of the present disclosure, it should be indicated that orientation or positional relationships indicated by the terms such as “center”, “upper”, “lower”, “left”, “right”, “vertical”, “horizontal”, “inner”, and “outer” are based on orientation or positional relationships as shown in the drawings, or orientation or positional relationships of a product of the present disclosure conventionally placed in use, merely for facilitating describing the present disclosure and simplifying the description, rather than indicating or implying that related device or elements have to be in the specific orientation, or configured or operated in a specific orientation, and thus they should not be construed as limitation to the present disclosure. Besides, the terms such as “first”, “second”, and “third” are merely used for distinguishing the description, but should not be construed as indicating or implying importance in the relativity.

Besides, the terms “include”, “contain” or any other variations thereof are intended to be non-exclusive, and thus a process, method, article or device including a series of elements not only include those elements, but also include other elements that are not listed definitely, or further include elements inherent to such process, method, article or device. Without more restrictions, an element defined with the wordings “including a . . . ” does not exclude presence of other same elements in the process, method, article or device including said element.

In addition, the terms such as “horizontal”, “vertical”, and “overhanging” do not mean that a component is required to be absolutely horizontal or overhanging, but may be slightly inclined. For example, by “horizontal” it merely means that a structure is more horizontal in comparison with “vertical”, rather than being completely horizontal, but slightly inclined.

In the description of the present disclosure, it should be further indicated that, unless otherwise specifically regulated and defined, the terms “provide”, “install”, “join”, and “connect” should be understood in a broad sense, for example, a connection may be a fixed connection, a detachable connection, or an integrated connection; it may be a mechanical connection or an electrical connection; it may be direct joining or indirect joining through an intermediary, and it also may be inner communication between two elements. The specific meanings of the above terms in the present disclosure could be understood by those skilled in the art according to specific situations.

Based on the above statement, as introduced in BACKGROUND ART, restricted by the penetration capability of the exemption-level photon quantum source (also called as isotope source), currently for large-diameter measurement scenes, the controlled radioactive sources with high energy and activity are generally required for measurement. However, the use of controlled radiation sources with high energy and activity is subject to many laws and regulations of “radiation safety management”. Therefore, using the exemption-level photon quantum source for high-precision measurement has become an urgent problem to be solved in the art.

Based on the discovery of the above technical problems, the inventors propose the following technical solutions through inventive efforts so as to solve or address the above problems. It should be noted that the above defects in the prior art scheme are solutions obtained by the inventors after practices and careful studies. Therefore, all of the discovery process of the above problems and the solutions in following embodiments of the present disclosure proposed for the above problems should be contributions made by the inventors to the present disclosure during creation of the invention, and should not be construed as technical contents well known to those skilled in the art.

In view of the above technical problems, the present embodiment provides a device for measuring in a large diameter. The device for measuring in a large diameter includes:
a measurement tube; and a mounting bracket located inside the measurement tube, wherein the mounting bracket is provided with a plurality of photon quantum sources, each photon quantum source is capable of emitting multiple kinds of photon quanta with different energies, and a gap for a to-be-measured petroleum to flow through is reserved between each photon quantum source and an inner wall of the measurement tube; an outer wall of the measurement tube is correspondingly provided with a photon quantum sensor for each photon quantum source, for detecting the multiple kinds of photon quanta emitted by corresponding photon quantum source. Since the photon quantum sources are fixed inside the measurement tube through the mounting bracket, the photon quanta generated by the photon quantum sources can easily penetrate the to-be-measured petroleum and be received by corresponding photon quantum sensors, and thus it is suitable for large-diameter measurement scenes.

In the above, a cross section of the measurement tube refers to a section perpendicular to an axis of the measurement tube, and the cross section thereof may be in any shape, for example, a circular shape, an oval shape, a rectangular shape, which is not specifically limited in the present embodiment. In addition, the device for measuring in a large diameter is not merely limited to the use for measuring the petroleum, but may also be used for measuring petroleum products such as crude oil, heavy oil, and finished oil, which is not specifically limited in the present embodiment. As an optional implementation mode, isotope Ba133 can be chosen as the photon quantum source, and the isotope Ba133 is capable of emitting photon quanta with energies of 31 k eV, 81 k eV, 356 k eV, and so on.

In the present embodiment, the mounting bracket includes: a mounting member, each photon quantum source being provided on the mounting member; and a fixing member, for fixedly connecting the mounting member to the inner wall of the measurement tube. In the above, a structure of the mounting member may be adaptively adjusted according to parameter information to be measured. For example, when a mass flow rate or a phase fraction of the to-be-measured petroleum is to be measured, the mounting bracket may be designed to be of different structures.

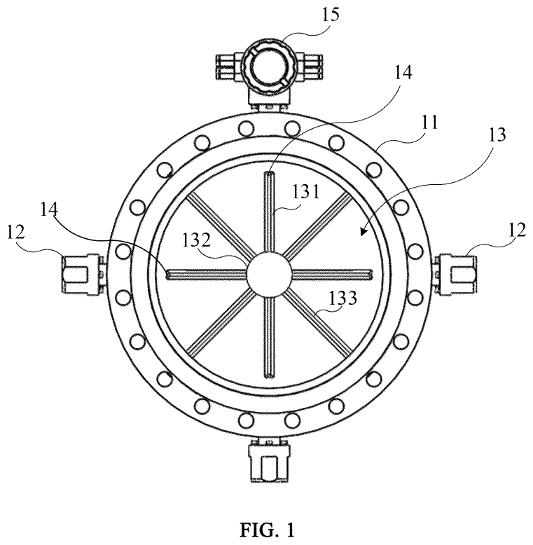

Taking phase fraction measurement as an example, as shown in FIG. 1, a mounting bracket 13 located inside a measurement tube 11 includes a mounting member and a fixing member 133, wherein the mounting member is used for providing photon quantum sources 14; and the mounting member is fixed on an inner wall of the measurement tube 11 by the fixing member 133, so that after a to-be-measured petroleum is introduced into the measurement tube 11, the mounting member can still remain stable. An outer wall of the measurement tube 11 is provided with, in positions corresponding to respective photon quantum sources 14, photon quantum sensors 12, for detecting multiple kinds of photon quanta generated by the photon quantum sources 14.

With continued reference to FIG. 1, the mounting member includes at least one mounting rod 131 and an arrangement member 132. The arrangement member 132 is arranged coaxially with the measurement tube 11. One end of each mounting rod 131 is connected to the arrangement member 132, and one end of each mounting rod 131 away from the arrangement member 132 is provided with one photon quantum source 14. When a plurality of photon quantum sources are provided, it is also necessary to provide a corresponding number of mounting rods, and the mounting rods are uniformly provided around the arrangement member, and in this way, the plurality of photon quantum sources 14 mounted on a plurality of mounting rods are radially distributed around the arrangement member 132, thereby being capable of more comprehensively measuring the water cut of the to-be-measured petroleum.

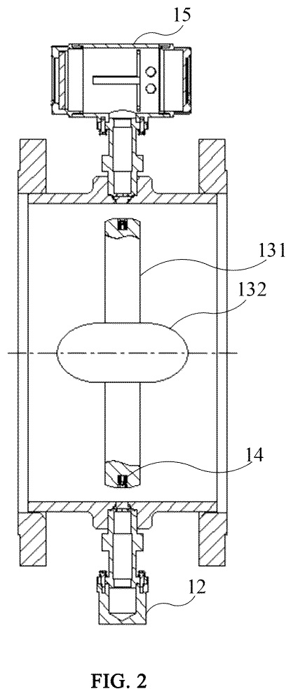

As shown in FIG. 2, it shows a longitudinal section of a device for measuring in a large diameter shown in FIG. 1. It can be seen that the arrangement member 132 presents a spindle structure with two small ends and thick middle, and the plurality of mounting rods 131 are uniformly distributed around an axis of the arrangement member 132. In this way, when measuring phase fraction information about the to-be-measured petroleum, the arrangement member 132 of the spindle structure can have a good flow guiding effect on the to-be-measured petroleum.

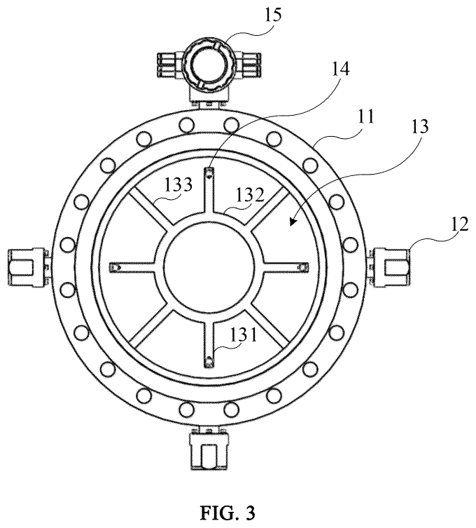

As shown in FIG. 3, the arrangement member 132 may also be shaped as a circular ring parallel to a cross section of the measurement tube 11, and the plurality of mounting rods 131 are likewise uniformly distributed around a periphery of the circular ring. Moreover, the photon quantum source is provided at one end of each mounting rod 131 away from the arrangement member 132.

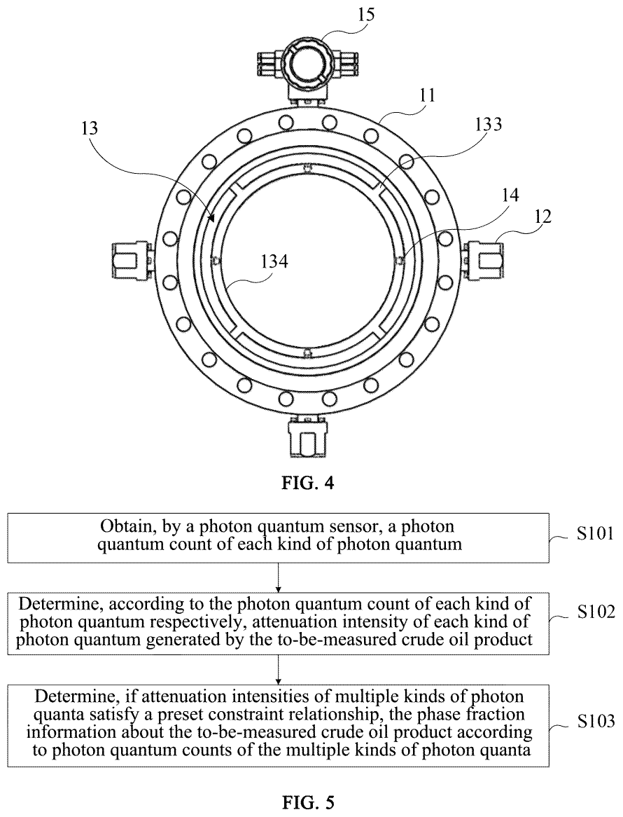

As shown in FIG. 4, as another implementation mode of the mounting member, the mounting member includes a mounting ring 134 parallel to the cross section of the measurement tube 11. The mounting ring 134 is fixed to an interior of the measurement tube 11 by the fixing member 133. All the photon quantum sources 14 are provided at a periphery of the mounting ring 134. Compared with FIG. 3, FIG. 4 omits the mounting rods 131 in FIG. 3, and thus resistance generated by the mounting rods 131 to the to-be-measured petroleum can be reduced.

Based on the above device for measuring in a large diameter, the present embodiment further provides a method for measuring petroleum in a large diameter. An electronic device for implementing this method may be the electronic device 15 in FIG. 1 to FIG. 4, wherein the electronic device 15 may be an embedded electronic device customized for petroleum measurement scenes. In order to make solutions provided in the present embodiment clearer, various steps of this method are described in detail below in conjunction with FIG. 5. But it should be understood that operations in the flow chart may not be implemented in order, and steps without context logical relationship may be reversed in order or simultaneously implemented. In addition, those skilled in the art, under the guidance of the contents of the present disclosure, could add one or more other operations to the flow chart, or remove one or more operations from the flow chart. As shown in FIG. 5, this method includes:
S101, obtaining, by a photon quantum sensor, a photon quantum count of each kind of photon quantum.

In this regard, with continued reference to FIG. 1, each photon quantum source 14 is capable of producing multiple kinds of photon quanta with different energies. Since the multiple kinds of photon quanta emitted by the photon quantum source need to pass through the to-be-measured petroleum so as to be detected by the photon quantum sensor 12, the to-be-measured petroleum will produce, to a certain extent, attenuation on the multiple kinds of photon quanta emitted by the photon quantum source. Studies find that the degree of attenuation of each kind of photon quantum is closely related to components in the to-be-measured petroleum. The petroleum is usually a miscible phase fluid, which includes water, associated natural gas, petroleum, tiny solid particles, etc., and these components produce different absorption effects on the photon quanta with various energies. Therefore, according to absorption degrees of the photon quanta with different energies, the phase fraction (for example, water cut and void fraction) and mixture densities in the to-be-measured petroleum can be calculated.

With continued reference to FIG. 5, based on the measured photon quantum count of each kind of quantum, the method for measuring petroleum in a large diameter provided in the present embodiment further includes:
S102, determining, according to the photon quantum count of each kind of photon quantum respectively, attenuation intensity of each kind of photon quantum generated by the to-be-measured petroleum.
S103, if attenuation intensities of multiple kinds of photon quanta satisfy a preset constraint relationship, determining the phase fraction information about the to-be-measured petroleum according to photon quantum counts of the multiple kinds of photon quanta.

In the above, the phase fraction information about the to-be-measured petroleum includes the water cut, the void fraction and the mixture densities of the to-be-measured petroleum, etc. Taking the water cut as an example, it should be understood that, currently in transition of petroleum, moisture analyzers used are mainly products based on capacitive and microwave/radio frequency measurement technologies, and its measurement accuracy can only reach a level of about 0.5%. Studies find that fluctuation of photon quantum intensity/photon quantum count for various energies in results measured by the photon quantum sensors is one of the principal factors affecting the measurement accuracy of the products. With regard to the above technical problems, further studies find that if the same to-be-measured petroleum is measured by multiple kinds of photon quanta with different energies, the attenuation intensities of the multiple kinds of photon quanta satisfy a specific constraint relationship, and just based on this constraint relationship, the present embodiment performs filtering processing on the measured photon quantum count of each kind of photon quantum. Therefore, in an optional implementation mode of step S103:

If the attenuation intensities of multiple kinds of photon quanta shows that the larger the energy of the photon quantum is, the smaller the attenuation is, then the electronic device determines the phase fraction information about the to-be-measured petroleum according to the photon quantum counts of the multiple kinds of photon quanta.

Exemplarily, it is further assumed that each photon quantum source is capable of emitting photon quanta with energy of 31 k eV, 81 k eV, or 356 k eV. For the to-be-measured petroleum, the generated attenuation intensity of the photon quantum of 31 k eV is expressed as a%; the generated attenuation intensity of the photon quantum of 81 k eV is expressed as b%; and the generated attenuation intensity of the photon quantum of 356 k eV is expressed as c%.

If a>b>c, it means that the attenuation intensities of the photon quanta with the three kinds of energies satisfy a preset constraint condition; otherwise, it is judged that the measured photon quantum counts are invalid. If the preset constraint condition is satisfied, the phase fraction information about the to-be-measured petroleum is further calculated further according to the photon quantum counts of the multiple kinds of photon quanta.

Taking the water cut as an example, the electronic device can obtain multiple water cuts of the to-be-measured petroleum according to the photon quantum counts of the multiple kinds of photon quanta; and determine an actual water cut of the to-be-measured petroleum according to the multiple water cuts. For example, a mean value of the multiple water cuts is taken as the actual water cut of the to-be-measured petroleum; or the multiple water cuts are ranked, and the water cut in a middle position is taken as the actual water cut of the to-be-measured petroleum. Certainly, more complex calculation may also be performed on the multiple water cuts, for example, the multiple water cuts are weighted so as to obtain the actual water cut, which is not specifically limited in the present embodiment.

In the present embodiment, based on the measured photon quantum counts of the multiple kinds of photon quanta, multiple water cuts are calculated by different water cut calculation methods. In an optional implementation mode, the electronic device can determine, according to the photon quantum count of each kind of photon quantum respectively, the water cut corresponding to the photon quantum count of each kind of photon quantum; then combine the photon quantum counts of the multiple kinds of photon quanta to obtain a plurality of photon quantum combinations; and determine, according to each photon quantum combination respectively, the water cut corresponding to each photon quantum combination.

It should be understood that, the method of calculating the water cut using the photon quantum count of a single kind of photon quantum is referred to as a single-energy mode, and the method of combining the photon quantum counts of multiple kinds of photon quanta in pairs and calculating the water cut according to combination results is referred to as a dual-energy method. These calculation methods are well-established algorithms in the art, and are not described in detail in the present embodiment.

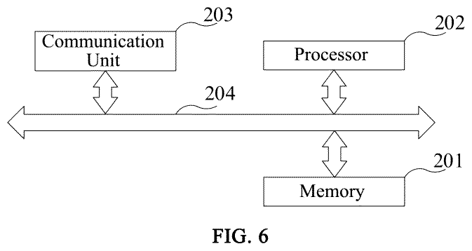

The present embodiment further provides an electronic device for implementing the above method. As shown in FIG. 6, the electronic device may include a processor 202 and a memory 201. Moreover, the memory 201 stores a computer program, and the processor implements, by reading and executing the computer program in the memory 201 corresponding to the above implementation mode, the method for measuring petroleum in a large diameter provided in the present embodiment.

With continued reference to FIG. 6, the electronic device further includes a communication unit 203. The memory 201, the processor 202 and the communication unit 203 are directly or indirectly electrically connected to each other by a system bus 204, so as to realize transmission or interaction of data.

In the above, this memory 201 may be an information recording device based on any electronic, magnetic, optical or other physical principle for recording an execution instruction, data, or the like. In some implementation modes, this memory 201 may be, but is not limited to, a volatile memory, a non-volatile memory, and a storage drive, etc.

In some implementation modes, the volatile memory may be a random access memory (RAM). In some implementation modes, the non-volatile memory may be a read only memory (ROM), a programmable read-only memory (PROM), an erasable programmable read-only memory (EPROM), an electric erasable programmable read-only memory (EEPROM), or a flash memory. In some implementation modes, the storage drive can be a disc driver, a solid state disk, a memory disk of any type (chg., optical disk and DVD), or a similar storage medium, or a combination thereof.

The communication unit 203 is configured to send and receive data through a network. In some implementation modes, the network may include a wired network, a wireless network, a fiber-optic network, a telecommunication network, an intranet, the Internet, a local area network (LAN), a wide area network (WAN), a wireless local area network (WLAN), a metropolitan area network (MAN), a wide area network (WAN), a public switched telephone network (PSTN), a bluetooth network, a ZigBee network, or a near field communication (NFC) network, or any combination thereof. In some implementation modes, the network may include one or more network access points. For example, the network may include a wired or wireless network access point, such as a base station and/or a network switching node, through which one or more components of a service request processing system may be connected to the network for exchanging data and/or information.

The processor 202 may be an integrated circuit chip having signal processing capability, and the processor may include one or more processing cores (for example, a single-core processor or a multi-core processor). By way of example only, the above processor may include a central processing unit (CPU), an application specific integrated circuit (ASIC), an application specific instruction-set processor (ASIP), a graphics processing unit (GPU), a physical processing unit (PPU), a digital signal processor (DSP), a field programmable gate array (FPGA), a programmable logic device (PLD), a controller, a microcontroller unit, reduced instruction set computer (RISC), a microprocessor, or the like, or any combination thereof.

It is to be understood that the structure shown in FIG. 6 is merely illustrative. The electronic device further may include more or fewer components than those shown in FIG. 6, or have a different configuration than that shown in FIG. 6. Various components shown in FIG. 6 can be realized by means of hardware, software, or a combination thereof.

In addition, the present embodiment further provides a measurement system, including the device for measuring in a large diameter and the electronic device in the above.

It should be understood that the device and the method disclosed in the above implementation modes also can be implemented in other modes. The device embodiments described above are merely exemplary, for example, the flow chart and the block diagram in the drawings show possible system architectures, functions and operations of the device, method and computer program product according to multiple embodiments of the present disclosure. In this regard, each block in the flow chart or block diagram may represent a module, part of a program segment or code, the module or the part of program segment or code includes one or more executable instructions for implementing a specified logical function. It should also be noted that, in some alternative implementations, the functions noted in the blocks may also occur out of the order noted in the drawings. For example, two continuous blocks may, in fact, be substantially concurrently executed, or they sometimes may also be executed in a reverse order, which depends upon the functionality involved. It is also to be noted that each block of the block diagram and/or flow chart, and combinations of blocks in the block diagram and/or flow chart, may be implemented in a dedicated hardware-based system that performs a specified function or action, or may be implemented in a combination of dedicated hardware and computer instructions.

The above-mentioned are merely for various embodiments of the present disclosure, but the scope of protection of the present disclosure is not limited thereto, and any change or substitution that may easily occur to those skilled in the present art within the technical scope disclosed in the present disclosure should be covered within the scope of protection of the present disclosure. Therefore, the scope of protection of the present disclosure should be based on the scope of protection of the claims.

What is claimed is:

1. A device for measuring petroleum in a large diameter, comprising:
a measurement tube; and
a mounting bracket, located inside the measurement tube, wherein the mounting bracket is provided with a plurality of photon quantum sources, and a gap for a to-be-measured petroleum to flow through is reserved between each of the photon quantum sources and an inner wall of the measurement tube; and
each of the photon quantum sources is capable of emitting multiple kinds of photon quanta, the multiple kinds of photon quanta have different energies, and an outer wall of the measurement tube is provided with photon quantum sensors for the respective photon quantum sources, for detecting the multiple kinds of photon quanta emitted by corresponding photon quantum sources.

2. The device for measuring petroleum in a large diameter according to claim 1, wherein the mounting bracket comprises:
a mounting member, wherein each of the photon quantum sources is provided on the mounting member; and
a fixing member, wherein the fixing member is configured for fixedly connecting the mounting member to the inner wall of the measurement tube.

3. The device for measuring petroleum in a large diameter according to claim 2, wherein the mounting member comprises at least one mounting rod and an arrangement member;
the arrangement member is arranged coaxially with the measurement tube; and
one end of each of the at least one mounting rod is connected to the arrangement member, and one end of each of the at least one mounting rod away from the arrangement member is provided with one of the photon quantum sources.

4. The device for measuring petroleum in a large diameter according to claim 2, wherein the mounting member comprises a mounting ring parallel to a cross section of the measurement tube; and
each of the photon quantum sources is provided at a periphery of the mounting ring.

5. A method for measuring petroleum in a large diameter, applied to the device for measuring petroleum in a large diameter according to claim 1, comprising steps of:
obtaining, by the photon quantum sensor, a photon quantum count of each kind of photon quantum;
determining, according to the photon quantum count of each kind of the photon quanta respectively, attenuation intensity of each kind of the photon quantum generated by the to-be-measured petroleum; and
determining, if attenuation intensities of the multiple kinds of photon quanta satisfy a preset constraint relationship, phase fraction information about the to-be-measured petroleum according to photon quantum counts of the multiple kinds of photon quanta.

6. The method for measuring petroleum in a large diameter according to claim 5, wherein the step of determining if the attenuation intensities of the multiple kinds of photon quanta satisfy a preset constraint relationship phase fraction information about the to-be-measured petroleum according to photon quantum counts of the multiple kinds of photon quanta comprises a step of:
determining, if the attenuation intensities of the multiple kinds of photon quanta shows that the larger the energy of the photon quantum is, the smaller the attenuation is, the phase fraction information about the to-be-measured petroleum according to the photon quantum counts of the multiple kinds of photon quanta.

7. The method for measuring petroleum in a large diameter according to claim 5, wherein the phase fraction information comprises an actual water cut of the to-be-measured petroleum, and the step of determining phase fraction information about the to-be-measured petroleum according to photon quantum counts of the multiple kinds of photon quanta comprises steps of:
obtaining multiple water cuts of the to-be-measured petroleum according to the photon quantum counts of the multiple kinds of photon quanta; and
determining the actual water cut of the to-be-measured petroleum according to the multiple water cuts.

8. The method for measuring petroleum in a large diameter according to claim 7, wherein the step of obtaining multiple water cuts of the to-be-measured petroleum according to the photon quantum counts of the multiple kinds of photon quanta comprises steps of:
determining, according to the photon quantum count of each kind of the photon quantum respectively, the water cut corresponding to the photon quantum count of each kind of the photon quantum;
combining the photon quantum counts of the multiple kinds of photon quanta to obtain a plurality of photon quantum combinations; and
determining, according to each of the photon quantum combinations respectively, the water cut corresponding to each of the photon quantum combinations.

9. An electronic device, comprising a processor and a memory, wherein the memory stores a computer program, and the computer program, when executed by the processor, implements the method for measuring petroleum in a large diameter according to claim 5.

10. The method for measuring petroleum in a large diameter according to claim 5, wherein the mounting bracket comprises:
a mounting member, wherein each of the photon quantum sources is provided on the mounting member; and
a fixing member, wherein the fixing member is configured for fixedly connecting the mounting member to the inner wall of the measurement tube.

11. The method for measuring petroleum in a large diameter according to claim 5, wherein the mounting member comprises at least one mounting rod and an arrangement member;
the arrangement member is arranged coaxially with the measurement tube; and
one end of each of the at least one mounting rod is connected to the arrangement member, and one end of each of the at least one mounting rod away from the arrangement member is provided with one of the photon quantum sources.

12. The method for measuring petroleum in a large diameter according to claim 5, wherein the mounting member comprises a mounting ring parallel to a cross section of the measurement tube; and
each of the photon quantum sources is provided at a periphery of the mounting ring.

13. The electronic device according to claim 9, wherein the step of determining if the attenuation intensities of the multiple kinds of photon quanta satisfy a preset constraint relationship phase fraction information about the to-be-measured petroleum according to photon quantum counts of the multiple kinds of photon quanta comprises a step of:
determining, if the attenuation intensities of the multiple kinds of photon quanta shows that the larger the energy of the photon quantum is, the smaller the attenuation is, the phase fraction information about the to-be-measured petroleum according to the photon quantum counts of the multiple kinds of photon quanta.

14. The electronic device according to claim 9, wherein the phase fraction information comprises an actual water cut of the to-be-measured petroleum, and the step of determining phase fraction information about the to-be-measured petroleum according to photon quantum counts of the multiple kinds of photon quanta comprises steps of:
obtaining multiple water cuts of the to-be-measured petroleum according to the photon quantum counts of the multiple kinds of photon quanta; and
determining the actual water cut of the to-be-measured petroleum according to the multiple water cuts.

15. The electronic device according to claim 9, wherein the step of obtaining multiple water cuts of the to-be-measured petroleum according to the photon quantum counts of the multiple kinds of photon quanta comprises steps of:
determining, according to the photon quantum count of each kind of the photon quantum respectively, the water cut corresponding to the photon quantum count of each kind of the photon quantum;
combining the photon quantum counts of the multiple kinds of photon quanta to obtain a plurality of photon quantum combinations; and
determining, according to each of the photon quantum combinations respectively, the water cut corresponding to each of the photon quantum combinations.
